(19) United States
(12) Patent Application Publication
(10) Pub. No.: US 2025/0281507 A1
(43) Pub. Date: Sep. 11, 2025

(54) METHODS OF TREATING NEPHROTIC SYNDROME

(71) Applicant: The Research Institute at Nationwide Children's Hospital, Columbus, OH (US)

(72) Inventor: William E. Smoyer, Bexley, OH (US)

(21) Appl. No.: 18/578,744

(22) PCT Filed: Jul. 12, 2022
(86) PCT No.: PCT/US2022/036789

§ 371 Date: Jan. 12, 2024

Related U.S. Application Data
(60) Provisional application No. 63/220,843, filed on Jul. 12, 2021.

(51) Int. Cl. A61K 31/573; A61K 31/4439; A61P 13/12

(52) U.S. Cl. CPC A61K 31/573; A61K 31/4439; A61P 13/12

(57) ABSTRACT

A method of treating nephrotic syndrome (NS) in a subject is described. The method includes administering a therapeutically effective amount of a PPARγ agonist to the subject.

### CROSS-REFERENCE TO RELATED APPLICATIONS

This application claims priority to U.S. Provisional Application Ser. No. 63/220,843, filed on Jul. 12, 2021, which is hereby incorporated by reference in its entirety.

### FIELD

This disclosure relates to the treatment of nephrotic syndrome. In particular, the disclosure provides a method of treating subjects suffering from NS with a PPARγ agonist.

### BACKGROUND

Nephrotic syndrome (NS) is one of the most common types of kidney diseases occurring in children. Children with NS typically present with massive proteinuria, edema and hypoalbuminaemia, which is almost always accompanied by marked hyperlipidemia. Even though NS is linked to various types of renal disease, the most common form (90%) identified in children is idiopathic NS, which progresses in the absence of any clinical features of primary extrarenal disorder (T.-S. Ha. Korean 1 Pediatr., vol. 60, no. 3, p. 55, 2017).

Clinically. NS can be classified into two general groups by the response to glucocorticoid therapy: steroid-sensitive NS (SSNS) and steroid-resistant NS (SRNS). Most patients with idiopathic NS initially respond well to glucocorticoids and enter the remission stage with a good renal prognosis. Such patients are referred to as having steroid-responsive or steroid-sensitive NS (SSNS). However, around 90% of these patients will relapse, with another half of those becoming steroid-dependent. The remaining 10% are recognized to have SRNS.

The majority of children who develop NS will experience at least one relapse of their disease. While many will have only infrequent relapses (≤3 relapses per year), a significant proportion (about 33%) of children who develop NS will experience multiple relapses. If a child has four or more relapses in any 12-month period, or two relapses in the first 6 months after diagnosis, he or she will be labeled as having frequent relapsing NS (FRNS). Some children respond to initial glucocorticoid treatment by entering clinical remission, but develop a relapse either while still receiving glucocorticoids, or within 2 weeks of discontinuation of treatment following a steroid taper. Such children may require continued low-dose treatment with glucocorticoids to prevent this rapid development of relapse. He or she will be labeled as having steroid-dependent NS (SDNS). Approximately 20% of children with NS are classified as having SDNS while about a further 20% are classified as having FRNS.

While the primary treatment of idiopathic NS has remained daily oral glucocorticoids for more than 60 years, new therapies are needed which limit relapse and/or prolonged exposure to glucocorticoids' inherent toxicities. Therefore, there remains a high unmet medical need to develop safer and more effective treatments for NS. The present disclosure is based on the discovery that PPARγ agonists, such as thiazolidinediones, are a particularly safe and effective therapy for the treatment of subjects, especially children, presenting with NS.

### SUMMARY OF THE INVENTION

In one aspect, the present disclosure provides a method of treating nephrotic syndrome (NS) in a subject (e.g., a pediatric subject) in need thereof comprising administering to said subject an effective amount of a PPARγ agonist (e.g., a thiazolidinedione).

In another aspect, the present disclosure provides a PPARγ agonist (e.g., a thiazolidinedione) for use in the treatment of nephrotic syndrome (NS) in a subject (e.g. a pediatric subject) in need thereof.

In another aspect, the present disclosure provides the use of a PPARγ agonist (e.g., a thiazolidinedione) in the manufacture of a medicament for the treatment of nephrotic syndrome (NS) in a subject (e.g., a pediatric subject) in need thereof.

In one embodiment of each of the above aspects, the subject (e.g., a pediatric subject) presents with steroid-dependent NS (SDNS) prior to initiation of treatment with a PPARγ agonist (e.g., a thiazolidinedione).

In one embodiment of each of the above aspects, the subject (e.g., a pediatric subject) presents with frequent relapsing NS (FRNS) prior to initiation of treatment with a PPARγ agonist (e.g., a thiazolidinedione).

In one embodiment of each of the above aspects, the PPARγ agonist (e.g., a thiazolidinedione) is co-administered to a subject (e.g., a pediatric subject) suffering from NS (including SDNS or FRNS) with an immunosuppressant, such as a glucocorticoid.

In one embodiment of each of the above aspects, the PPARγ agonist (e.g., a thiazolidinedione) produces a “steroid-sparing effect” when co-administered with a glucocorticoid to treat a subject (e.g., a pediatric subject) suffering from NS (including SDNS or FRNS).

In one embodiment of each of the above aspects, when co-administered with a PPARγ agonist (e.g., a thiazolidinedione) to treat a subject (e.g., a pediatric subject) suffering from NS (including SDNS or FRNS), the amount of the immunosuppressive medication (e.g., glucocorticoid) required is less than the amount needed for effective treatment when the immunosuppressive medication is administered as a monotherapy.

### BRIEF DESCRIPTION OF THE FIGURES

The present invention may be more readily understood by reference to the following drawings, wherein:

FIG. 1 shows in (A) a time course of change in proteinuria for all eight patients before (shaded) and after (unshaded) the addition of pioglitazone to their treatment regimen, and shows in (B) a time course of change in proteinuria for Responders (red dashed line) and Non-responders (blue dotted line) before (shaded) and after (unshaded) the addition of pioglitazone to their treatment regimen.

FIG. 2 shows in (A) a time course of change in serum albumin for all eight patients before (shaded) and after (unshaded) the addition of pioglitazone to their treatment regimen, and shows in (B) a time course of change in serum albumin for Responders (red dashed line) and Non-responders (blue dotted line) before (shaded) and after (unshaded) the addition of pioglitazone to their treatment regimen.

FIG. 3 shows in (A) a time course of changes in Total Immunosuppression Score (TIS) for all eight patients before and after the addition of pioglitazone to their treatment regimen, and shows in (B) a time course of changes in TIS for Responders (red dashed line) and Non-responders (blue dotted line) before (shaded) and after (unshaded) the addition of pioglitazone to their treatment regimen.

FIG. 4 shows a time course of clinical parameters for each of the eight patients over five time periods before (shaded) and five time periods after (unshaded) the addition of pioglitazone to their treatment regimen. Urine protein to creatinine ratios are shown as blue triangles. Serum Albumin levels are shown as green squares. Total immunosuppression scores are shown as red diamonds.

### DETAILED DESCRIPTION OF THE INVENTION

The present disclosure provides a method of treating nephrotic syndrome (NS) in a subject is described. The method includes administering a therapeutically effective amount of a PPARγ agonist to the subject. In one aspect, the invention is based on the discovery that TZDs, particularly pioglitazone, can enhance the effects of GCs on NS in pediatric subjects, including in children presenting with steroid-dependent nephrotic syndrome (SDNS) or frequent relapsing nephrotic syndrome (FRNS). It has been found that TZDs can enhance the proteinuria-reducing effects of GCs during NS therapy, including in children presenting with SDNS or FRNS.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

The term “therapeutically effective amount” or “effective amount” refers to the amount of an active agent administered to the subject that elicits a biological or medicinal response that is sought by a researcher, healthcare provider or individual in accordance with the present disclosure on the treated subject.

The terms “subject,” “individual” and “patient” are used interchangeably herein, and refer to a human. The subject treated according to the present disclosure may be an adult, pediatric, or elderly subject.

The terms “pediatric subject”, “child” or “children” mean a subject between the ages of about 12 months and about 17 years. It should be understood, that the terms “a method of treating NS in a pediatric subject” and “a method of treating NS in children” includes (i) treating a subject who is between the ages of about 12 months and about 17 years throughout the course of treatment and (ii) treating a subject who is between the ages of about 12 months and about 17 years at the initiation of the course of treatment, but who is older than about 17 years at the conclusion of the course of treatment. In some embodiments, the subject has an age of 17 years or less, in further embodiments the subject has an age of 12 or less, while in further embodiments the subject has an age of 3 years or less. In one example, a subject is a pediatric subject who is aged 1-17 years at initiation of therapy (e.g., TZD therapy).

The terms “treat” and “treatment” refer herein to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition. “Treatment” can also mean prolonging survival as compared to expected survival if not receiving treatment. Those in need of treatment include those already with the disease or condition as well as those prone to having the disease or condition or those in which the disease or condition is to be prevented.

The term “in need of treatment” and the term “in need thereof” when referring to treatment ae used interchangeably and refer to a judgment made by a caregiver (e.g., physician, nurse, nurse practitioner) that a patient will benefit from treatment, or a subject who has been identified as having an increased risk of having or developing nephrotic syndrome.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires, in particular, where the indefinite article is used, the specification is to be understood as contemplating plurality as well as singularity, unless the context requires otherwise.

In one aspect, the present invention provides a method of treating nephrotic syndrome (NS) in a subject. The method includes administering to the subject a therapeutically effective amount of a PPARγ agonist.

### Nephrotic Syndrome

Nephrotic syndrome (NS) is considered to be the most prevalent glomerular disease of childhood, with an incidence of around 2 in 100,000 children. Nephrotic syndrome is a general term that refers to the loss of protein in the urine (proteinuria), hyperlipidemia (hypercholesterolemia and hypertriglyceridemia), and edema. Nephrotic syndrome involves changes in the pathology of cells in the kidney, such as podocytes.

Childhood NS is most often due to primary glomerulopathies. These include minimal change nephrotic syndrome (MCNS), focal segmental glomerulosclerosis (FSGS), mesangial proliferative glomerulonephritis, membranoproliferative glomerulonephritis (MPGN) and membranous nephropathy (MN). FSGS is described as scarring of the glomerulus that includes several distinct changes, where only a segment of the glomerulus and some, but not all, glomeruli are affected. Progression to end-stage renal disease (ESRD) is closely correlated with the development of FSGS. While the historical classification of FSGS separated this condition into primary FSGS (i.e., idiopathic) versus secondary FSGS (i.e., due to known causes), integration of more robust clinical, histologic, and genetic knowledge of this disease, has led to the proposal that FSGS can be categorized into six forms: (1) primary FSGS, (2) adaptive FSGS, (3) APOL1 FSGS, (4) genetic FSGS, (5) virus-associated FSGS, and (6) medication-/toxin-associated FSGS. References in the present disclosure to the treatment of FSGS includes treatment of any one of the six forms of FSGS discussed herein.

The present invention provides a method of treating nephrotic syndrome (NS) in a subject. In some embodiments, the nephrotic syndrome is steroid-dependent nephrotic syndrome (SDNS). In some embodiments, the nephrotic syndrome is frequent relapsing nephrotic syndrome (FRNS).

In an additional aspect, the present invention provides a method of treating of focal segmental glomerulosclerosis (FSGS) in a subject (e.g., a pediatric subject) by administering an effective amount of a PPARγ agonist (e.g., a thiazolidinedione).

The present disclosure also relates to the treatment of minimal change nephrotic syndrome (MCNS) in a subject (e.g. a pediatric subject) by administering an effective amount of a PPARγ agonist (e.g., a thiazolidinedione) to the subject.

The present disclosure also relates, in one aspect, to the treatment of membranous nephropathy (MN) in a subject (e.g. a pediatric subject) by administering an effective amount of a PPARγ agonist (e.g., a thiazolidinedione) to the subject.

For treatment of any of the types of nephrotic syndrome described herein, the subject (e.g., pediatric subject) may present with symptoms prior to initiation of administration of the PPARγ agonist, such as a TZD (e.g., pioglitazone).

A variety of signs and symptoms are known to be associated with nephrotic syndrome. These include respiratory tract infection, allergy, macrohematuria, symptoms of infection, hypotension, respiratory distress, tachypnea, seizure, anorexia, irritability, fatigue, and disarrhea. Nephrotic syndrome can be diagnosed using urinalysis, urine protein quantification, serum albumin quantification, and a lipid panel. With the advent of electron microscopy, the changes now known as the hallmarks for the disease include diffuse loss of podocyte foot processes, vacuolation of the podocyte foot processes, and growth of microvilli on the visceral epithelial cells. Diabetic nephropathy is the most common cause of nephrotic syndrome.

Nephrotic syndrome, or nephrosis, is defined by the presence of nephrotic-range proteinuria, edema, hyperlipidemia, and hypoalbuminemia. While nephrotic-range proteinuria in adults is characterized by protein excretion of 3.5 g or more per day, in children it is defined as protein excretion of more than 40 mg/m²/h or a first-morning urine protein/creatinine of 2-3 mg/mg creatinine or greater. In some embodiments, the nephrotic syndrome is pediatric nephrotic syndrome, which is the occurrence of pediatric nephrotic syndrome in a child.

### PPARγ Agonists

The term “PPARγ” means Peroxisome Proliferator-Activated Receptor gamma. PPARγ is an orphan member of the steroid/thyroid/retinoid receptor superfamily of ligand-activated transcription factors. PPARγ is one of a sub-family of closely related PPARs encoded by independent genes (C. Dreyer et. al., Cell (1992) 68:879-887; A. Schmidt et al., Mol. Endocrinol. (1992) 6:1634-1641; Y. Zhu et al., J. Biol. Chem. (1993) 268:26817-26820; S. A. Kliewer et al., Proc. Nat. Acad. Sci. USA (1994) 91:7355-7359). Three mammalian PPARs have been isolated and termed PPAR-alpha, PPAR-gamma, and NUC-1 (also known as PPAR-delta). These PPARs regulate expression of target genes by binding to DNA sequence elements, termed PPAR response elements (PPRE).

In some embodiments, the PPARγ agonist is a thiazolidinedione (TZD). The term “thiazolidinedione” (also known as “glitazones” and “TZDs”) means a class of heterocyclic compounds containing of a thiazole ring that have historically been used to treat insulin resistance in type II diabetes. TZDs work by activating PPARs, with greatest specificity for PPARγ. Clinically, TZDs have also been shown to reduce albuminuria in type 2 diabetes, as well as proteinuria in non-diabetic kidney diseases. Members of the thiazolidinedione class of drugs that have been marketed include pioglitazone (ACTOS®), rosiglitazone (AVANDIA®), lobeglitazone (DUVIE®), and troglitazone (REZULIN®). Other members of the thiazolidinedione class of drugs include netoglitazone, rivoglitazone and ciglitazone. References herein to the treatment of NS using a “thiazolidinedione” or “TZD” includes the treatment of NS using an active metabolite of a “thiazolidinedione” or “TZD” having PPARγ agonist activity. Of particular interest are metabolites of pioglitazone, including metabolites described by T. Jaakkola et al. in Basic & Clinical Pharmacology (2006), 99, pages 44-51.

Exemplary thiazolidinediones (TZDs) for use in a method of the present disclosure are selected from the group consisting of pioglitazone, rosiglitazone, lobeglitazone, ciglitazone, darglitazone, englitazone, netoglitazone, rivoglitazone, troglitazone, and balaglitazone. Pioglitazone is a preferred TZD for use in a method of the present disclosure.

Pioglitazone is a thiazolidinedione compound that is typically used to treat diabetes. It has the structure shown in formula I below:

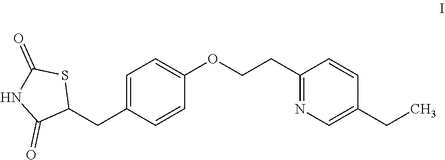 [SMILES: CCc1ccc(CCOc2ccc(CC3SC(=O)NC3=O)cc2)nc1]

In addition to their use in combination with immunosuppressive medicaments to treat NS, as described herein, PPARγ agonists, such as TZDs, also provide effective monotherapy to treat subjects (e.g. pediatric subjects) with NS. Therefore, in another aspect, the present disclosure provides method of treating nephrotic syndrome in a subject (e.g., a pediatric subject) by administering an effective amount of a PPARγ agonist, such as a thiazolidinedione.

The specific therapeutically effective dose level of the PPARγ agonist for any particular subject will depend upon a variety of factors, including the level of severity of NS, the activity of the specific compound employed, the age, body weight and general health of the patient, the time and duration of administration, whether or not other therapeutics are being administered, and like factors well known in the medical arts. However, in general, a therapeutic dose of a TZD, such as pioglitazone, may be in the dose range of about 1 mg to about 100 mg QD, e.g., about 5 mg to about 50 mg QD, e.g., about 5 mg, about 10 mg, about 15 mg, about 20 mg, about 25 mg, about 30 mg, about 35 mg, about 40 mg, about 45 mg or about 50 mg. In one embodiment, pioglitazone is administered orally to a pediatric subject at a dose of about 15-45 mg QD (i.e., per day).

Treatment with Immunosuppressive Medication

In some embodiments, the subject is also treated with one or more immunosuppressive medications. In some embodiments, the PPAR-γ agonist and the immunosuppressive mediation are administered concomitantly. Concomitant, as used herein, means that the two drugs are administered either in combination or that the PPAR-γ agonist is administered separately while the immunosuppressive medication is present in a therapeutically effective amount. As described herein, the PPAR-γ agonists such as pioglitazone enhance the proteinuria-reducing effects of glucocorticoids. Therefore, when administered concomitantly, the therapeutically effective amount of immunosuppressive mediation is substantially less than would be required in the absence of the PPAR-γ agonist.

Immunosuppressive medications which may also be administered herein to treat NS include particularly glucocorticoids. Glucocorticoids include, for example, aldosterone, beclomethasone, betamethasone, budesonide, cloprednol, cortisone, cortivazol, eoxycortone, desonide, desoximetasone, difluorocortolone, luclorolone, flumethasone, flunisolide, fluocinolone, luocinonide, fluocortin butyl, fluorocortisone, fluorocortolone, fluorometholone, flurandrenolone, fluticasone, alcinonide, hydrocortisone, comethasone, meprednisone, methylprednisolone, mometasone, paramethasone, prednisolone, prednisone, tixocortol, triamcinolone, and others, and their respective pharmaceutically acceptable derivatives, such as beclomethasone diproprionate, dexamethasone 21-isonicotinate, fluticasone propionate, icomethasone enbutate, tixocortol 21-pivalate, triamcinolone acetonide, and others.

In some embodiments, the glucocorticoid is selected from prednisone, prednisolone, methylprednisolone and dexamethasone. In further embodiments, the glucocorticoid is prednisone. In some subjects with more severe NS, other immunosuppressive medications may be co-administered with a glucocorticoid. Exemplary immunosuppressive medications include a cyclosporin (e.g., cyclosporin A), cyclophosphamine, a calcineurin inhibitor, tacrolimus, rituximab, or mycophenolate mofetil.

The therapeutic dosage regimen of the immunosuppressive medicament(s) for use according to the present disclosure prior to initiation of PPARγ agonist therapy will be the regimen that is routinely used to treat subjects with NS, adjusted as necessary by the medical practitioner based on the age and weight of the subject and the severity of the condition. Following initiation of PPARγ agonist therapy, the dose of the immunosuppressive medicament(s) may need to be suitably adjusted depending upon how the subject responds to the addition of the PPARγ agonist to the treatment regimen.

Therefore, in one aspect, the present disclosure provides a combination therapy for the treatment of NS in subjects (especially children) comprising at least one immunosuppressive medication and a PPARγ agonist, e.g., a thiazolidinedione (TZD). In a further embodiment, the combination therapy for the treatment of NS in subjects (especially children) comprises a glucocorticoid (e.g., selected from prednisone, prednisolone, methylprednisolone and dexamethasone) and a PPARγ agonist, e.g. a TZD. In a yet further embodiment, the combination therapy for the treatment of NS in subjects (especially children) comprises a glucocorticoid (e.g. selected from prednisone, prednisolone, methylprednisolone and dexamethasone) and pioglitazone.

In one embodiment, the addition of a PPARγ agonist, such as a TZD (e.g., pioglitazone), to the immunosuppressive medicament(s) drug regimen for the treatment of NS in subjects (especially children) results in an enhanced therapeutic effect, such as a further lowering of proteinuria levels, compared to use of the immunosuppressive medicament(s) alone. In some embodiments, the PPARγ agonist and the one or more immunosuppressive medications act synergistically to treat NS.

Other drugs commonly used to treat NS may also be co-administered to the subject, including blood pressure medications (e.g., lisinopril, benazepril, captopril or enalapril), diuretics (e.g., furosemide, spirolactone, hydrochlorothiazide or metolazone), cholesterol-reducing medications (e.g., statins such as atorvastatin, Fluvastatin, lovastatin, pravastatin, rosuvastatin or simvastatin) or blood thinners (e.g., heparin, warfarin, dabigatran, apixaban or rivaroxaban).

### Beneficial Effects of Treatment

Treatment of nephrotic syndrome in a subject by administering an effective amount of a PPARγ agonist to the subject can provide a wide variety of different benefits. An effective therapy for treating NS in children, including in children presenting with SDNS or FRNS, comprises administering to the pediatric subject currently treated with one or more immunosuppressive medications, such as a low-dose GC or high-dose GC, a TZD (e.g., pioglitazone) in a suitable dosage amount. The enhanced lowering of proteinuria levels achieved in pediatric subjects when adding a TZD to the treatment regimen allows for a reduction in the dose of the immunosuppressive medication(s) administered, such as a reduction in the dose of a GC, without any loss in efficacy. Thus, when adding a TZD to the treatment regimen, an immunosuppressive medication (e.g., GC) “sparing effect” is achieved, which can lead to subsequent reductions in overall immunosuppression and an improved side effect profile.

In some embodiments, administering a PPARγ agonist to the subject produces a reduction in proteinuria in the subject. Proteinuria indicates an elevated presence of protein the urine of a subject, with normal excretion being <150 mg/d. In some embodiments, the reduction in proteinuria in a subject can range from a reduction of about 5% to a reduction of about 100%, while in other embodiments the reduction in proteinuria ranges from about 10% to about 90%, while in further embodiments the reduction in proteinuria ranges from about 20% to about 80%, while in a yet further embodiment the reduction in proteinuria ranges from about 30% to about 70%. The reduction in proteinuria can be achieved within 1 week, within 2 weeks, within one month, within two months, within three months, or within 5-7 months of the initiation of PPARγ agonist therapy.

In some embodiments, >30% reduction in proteinuria is achieved within 1 month following initiation of PPARγ agonist therapy. In further embodiments, a >40% reduction in proteinuria is achieved within 1 month following initiation of PPARγ agonist therapy. In yet further embodiments, a >50% reduction in proteinuria is achieved within 1 month following initiation of PPARγ agonist therapy. In additional embodiments, a >60% reduction in proteinuria is achieved within 1 month following initiation of PPARγ agonist therapy.

In some embodiments, protein levels have been normalized (i.e., proteinuria has been eliminated) within 5-7 months following initiation of PPARγ agonist therapy. In other embodiments, a >30% reduction in proteinuria is achieved within 5-7 months following initiation of PPARγ agonist therapy. In further embodiments, a >40% reduction in proteinuria is achieved within 5-7 months following initiation of PPARγ agonist therapy. In yet further embodiments, a >50% reduction in proteinuria is achieved within 5-7 months following initiation of PPARγ agonist therapy. In additional embodiments, >60% reduction in proteinuria is achieved within 5-7 months following initiation of PPARγ agonist therapy. In yet further embodiments, a >95% reduction in proteinuria is achieved within 5-7 months following initiation of PPARγ agonist therapy.

In some embodiments, administering the PPARγ agonist increases the level of serum albumin in the subject. Albumin is a plasma protein normally found in very small quantities in the urine. Albuminuria is an abnormal loss of albumin in the urine. In some embodiments, administering the PPARγ agonist achieves a >40% mean increase in serum albumin within 1 month following initiation of therapy. In further embodiments, administering the PPARγ agonist achieves a >75% mean increase in serum albumin is achieved within 5-7 months following initiation of therapy.

In some embodiments, such as when the PPARγ agonist is administered in combination with an immunosuppressant, administering the PPARγ agonist produces an immunosuppressive medication sparing effect. A sparing effect, as used herein, refers to the ability of one compound (e.g., a drug such as a PPARγ agonist) to decrease the amount of another drug to achieve the same effect. In other words, is “spares” the subject from an otherwise higher necessary dose. Accordingly, the one or more immunosuppressive medications is/are administered at a reduced dose compared to standard therapy. Administering a PPARγ agonist to a subject can result in a reduction of >10%, >20%, >30%, >40%, or greater than >50% of the immunosuppressant required. In some embodiments, a >40% reduction in immunosuppressant is achieved following PPARγ agonist therapy.

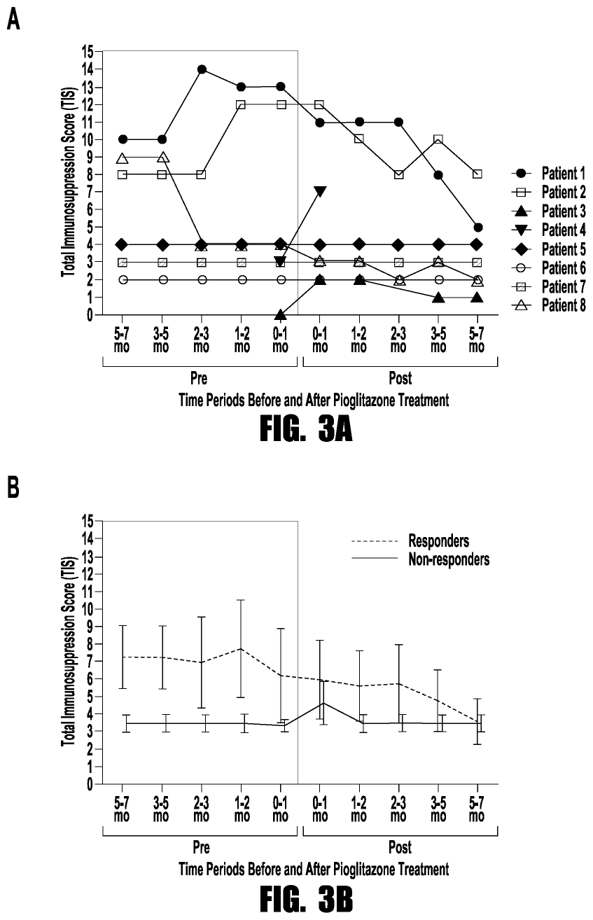

The reduction in overall immunosuppression can be quantified by comparing the “Total Immunosuppression Score” (TIS) prior to and after the addition of a TZD to the drug regimen (see FIG. 3 herein). The TIS is based on a combination of the number of immunosuppressive drugs being administered and the dose at which each drug is administered. The score for each drug is on a scale of 0 to 4, where: 0=no drug administered; 1=drug administered up to less than 25% of the standardized full dose; 2=drug administered between 25% and less than 50% of the standardized full dose; 3=drug administered between 50% and less than 75% of the standardized full dose; 4=drug administered between 75% and 100% of the standardized full dose. As an example, if five different immunosuppressive medicaments are co-administered to a subject each at their standardized full dose, the TIS=20.

Sparing effects can also be achieved when the PPARg agonist is administered in combination with other drugs. Administering a PPARγ agonist to a subject can result in a reduction of >10%, >20%, >30%, >40%, or greater than >50% of the immunosuppressant required. In some embodiments, administering a PPARγ agonist produces a glucocorticoid sparing effect, while in further embodiments, administering a PPARγ agonist produces a prednisone sparing effect.

In some embodiments, nephrotic syndrome (NS) is treated by administering a PPARγ agonist, either alone or in combination with other active agents, without any consequential adverse effects. For example, in some embodiments, NS is treated without any consequential hypoglycemia, while in further embodiments. NS may be treated with a PPARγ agonist, such as pioglitazone, without any consequential hypoglycemia.

In some embodiments, administering the PPARγ agonist produces an improvement in Patient Reported Outcomes (PROs), including measures of physical health, mental health, social health, or global health, in the subject. In further embodiments, the NS is treated with an accompanying reduction in hospitalizations. In a yet further embodiment, NS may be treated with a PPARγ agonist, such as pioglitazone, resulting in an accompanying reduction in hospitalizations.

One particular advantage of the present disclosure is that, using a PPARγ agonist such as pioglitazone. NS can be treated without subjects experiencing any significant consequential physical, mental or social health issues, for example, as listed in PROMIS® (the Patient-Reported Outcomes Measurement Information System funded by the NIH). Thus, for example, subjects did not report worsening edema, pain (including pain intensity and interference), fatigue and lack of physical activity, sleep issues, GI symptoms, dyspnea, depression, anxiety, psychosocial illness or relationship issues with family or peers. Indeed, subjects showed an improvement in Patient Reported Outcomes (PROs) following the administration of a PPARγ agonist such as pioglitazone.

In one embodiment, treatment of NS with a PPARγ agonist, such as pioglitazone, produces an improvement in Patient Reported Outcomes (PROs), including measures of physical health, mental health, social health, or global health, in the subject.

In some embodiments, nephrotic syndrome (NS) is treated without any worsening edema. In further embodiments, treatment of NS further comprises an improvement (i.e., a decrease) in edema. In some embodiments. NS may be treated with a PPARγ agonist, such as pioglitazone, resulting in an accompanying improvement in edema.

Nephrotic syndrome (NS) is characterized by glomerular injury and massive urinary protein loss, which leads to a severe, acquired hypercoagulopathy associated with an elevated risk for life-threatening venous thromboembolic (VTE) disease that afflicts up to 25% of adult and 3% of childhood NS patients. The present disclosure also provides, in one aspect, the use of a PPARγ agonist, such as a TZD, to alleviate hypercoagulopathy in adult or pediatric subjects with NS. In another aspect, the present disclosure provides the use of a PPARγ agonist, such as a TZD, to reduce NS-associated hypercoagulopathy-mediated VTE risk. In a further aspect, a PPARγ agonist, such as a TZD, and a glucocorticoid are co-administered to alleviate hypercoagulopathy in adult or pediatric subjects with NS. In another aspect, a PPARγ agonist, such as a TZD, and a glucocorticoid are co-administered to reduce NS-associated hypercoagulopathy-mediated VTE risk.

### Exemplary Embodiments

A number of exemplary embodiments are provided herein:

Embodiment 1: A method of treating nephrotic syndrome (NS) in a subject (e.g. a pediatric subject), comprising administering to said subject an effective amount of a PPARγ agonist such as a thiazolidinedione (TZD).

Embodiment 2: A method of treating steroid-dependent nephrotic syndrome (SDNS) in a subject (e.g. a pediatric subject), comprising administering to said subject an effective amount of a PPARγ agonist such as a thiazolidinedione (TZD).

Embodiment 3: A method of treating frequent relapsing nephrotic syndrome (FRNS) in a subject (e.g. a pediatric subject. e.g., aged 1-17 years), comprising administering to said subject an effective amount of a PPARγ agonist such as a thiazolidinedione (TZD) (e.g., at initiation of TZD therapy).

Embodiment 4: The method according to any one of Embodiments 1-3, wherein the subject (e.g. pediatric subject) has focal segmental glomerulosclerosis.

Embodiment 5: The method according to any one of Embodiments 1-3, wherein the subject (e.g. pediatric subject) has collapsing focal segmental glomerulosclerosis.

Embodiment 6: The method according to any one of Embodiments 1-3, wherein the subject (e.g. pediatric subject) has minimal change nephrotic syndrome.

Embodiment 7: The method according to any one of Embodiments 1-6, wherein the subject (e.g. pediatric subject) is also being treated with one or more immunosuppressive medications.

Embodiment 8: The method according to Embodiment 7, wherein an immunosuppressive medication is a glucocorticoid, such as prednisone, prednisolone, methylprednisolone and dexamethasone.

Embodiment 9: The method according to any one of Embodiments 7-8, wherein the PPARγ agonist and the one or more immunosuppressive medications act synergistically to treat NS.

Embodiment 10: The method according to any one of Embodiments 7-8, wherein the PPARγ agonist and the one or more immunosuppressive medications act synergistically to treat SDNS.

Embodiment 11: The method according to any one of Embodiments 7-8, wherein the PPARγ agonist and the one or more immunosuppressive medications act synergistically to treat FRNS.

Embodiment 12: The method according to Embodiment 7 or 8, wherein the one or more immunosuppressive medications is/are administered at a reduced dose compared to standard therapy.

Embodiment 13: The method according to any one of Embodiments 7-12, wherein administering the PPARγ agonist (e.g. TZD) produces a immunosuppressive medication sparing effect.

Embodiment 14: The method according to any one of Embodiments 1-13, wherein the PPARγ agonist (e.g. TZD) is selected from the group consisting of pioglitazone, rosiglitazone, lobeglitazone, ciglitazone, darglitazone, englitazone, netoglitazone, rivoglitazone, troglitazone, and balaglitazone.

Embodiment 15: The method according to any one of Embodiments 1-14, wherein the PPARγ agonist (e.g. TZD) is pioglitazone.

Embodiment 16: The method according to Embodiment 15, wherein pioglitazone is administered orally to the subject (e.g. pediatric subject) at a dose of about 15-45 mg QD.

Embodiment 17: The method according to any one of Embodiments 1-16, wherein administering the PPARγ agonist produces a reduction in proteinuria in the subject.

Embodiment 18: The method according to Embodiment 17, wherein >30% reduction in proteinuria is achieved within 1 month following initiation of PPARγ agonist therapy.

Embodiment 19: The method according to Embodiment 17, wherein >40% reduction in proteinuria is achieved within 1 month following initiation of PPARγ agonist therapy.

Embodiment 20: The method according to Embodiment 17, wherein >50% reduction in proteinuria is achieved within 1 month following initiation of PPARγ agonist therapy.

Embodiment 21: The method according to Embodiment 17, wherein >60% reduction in proteinuria is achieved within 1 month following initiation of PPARγ agonist therapy.

Embodiment 22: The method according to Embodiment 17, wherein >30% reduction in proteinuria is achieved within 5-7 months following initiation of PPARγ agonist therapy.

Embodiment 23: The method according to Embodiment 17, wherein >40% reduction in proteinuria is achieved within 5-7 months following initiation of PPARγ agonist therapy.

Embodiment 24: The method according to Embodiment 17, wherein >50% reduction in proteinuria is achieved within 5-7 months following initiation of PPARγ agonist therapy.

Embodiment 25: The method according to Embodiment 17, wherein >60% reduction in proteinuria is achieved within 5-7 months following initiation of PPARγ agonist therapy.

Embodiment 26: The method according to Embodiment 17, wherein >95% reduction in proteinuria is achieved within 5-7 months following initiation of PPARγ agonist therapy.

Embodiment 27: The method according to Embodiment 17, wherein normalization of proteinuria is achieved within 5-7 months following initiation of PPARγ agonist therapy.

Embodiment 28: The method according to any one of Embodiments 1-27, wherein administering the PPARγ agonist produces an increase in serum albumin in the subject.

Embodiment 29: The method according to Embodiment 28, wherein >40% mean increase in serum albumin is achieved within 1 month following initiation of PPARγ agonist therapy.

Embodiment 30: The method according to Embodiment 28, wherein >75% mean increase in serum albumin is achieved within 5-7 months following initiation of PPARγ agonist therapy.

Embodiment 31: The method according to any one of Embodiments 7-30, wherein administering the PPARγ agonist produces a immunosuppressive medication sparing effect.

Embodiment 32: The method according to Embodiment 31, wherein >40% reduction in total immunosuppression is achieved following PPARγ agonist therapy.

Embodiment 33: The method according to Embodiment 31, wherein administering a PPARγ agonist produces a glucocorticoid sparing effect.

Embodiment 34: The method according to Embodiment 31, wherein administering a PPARγ agonist produces a prednisone sparing effect.

Embodiment 35: The method according to any one of Embodiments 17-34, wherein the subject is a pediatric subject.

Embodiment 36: The method according to any one of Embodiments 17-35, wherein the PPARγ agonist is a thiazolidinedione (TZD).

Embodiment 37: The method according to Embodiment 36, wherein the TZD is selected from the group consisting of pioglitazone, rosiglitazone, lobeglitazone, ciglitazone, darglitazone, englitazone, netoglitazone, rivoglitazone, troglitazone, and balaglitazone.

Embodiment 38: The method according to Embodiment 36, wherein the TZD is pioglitazone.

Embodiment 39: The method according to any one of Embodiments 1-38, wherein NS is treated without any consequential adverse effects.

Embodiment 40: The method according to any one of Embodiments 1-39, wherein administering the PPARγ agonist produces an improvement in Patient Reported Outcomes (PROs), including measures of physical health, mental health, social health, or global health, in the subject.

Embodiment 41: The method according to any one of Embodiments 1-40, wherein NS is treated without any worsening edema.

Embodiment 42: The method according to any one of Embodiments 1-41, wherein NS is treated with an accompanying improvement in edema.

Embodiment 43: The method according to any one of Embodiments 1-42, wherein NS is treated with an accompanying reduction in hospitalizations.

Embodiment 44: The method according to any one of Embodiments 1-43, wherein NS is treated without any consequential hypoglycemia.

Embodiment 45: The method according to any one of Embodiments 1-44, wherein administering a PPARγ agonist reduces NS-associated hypercoagulopathy-mediated VTE risk.

Embodiment 46: A PPARγ agonist (e.g. TZD) for use in the treatment of nephrotic syndrome (NS) in a subject (e.g. pediatric subject) in need thereof.

Embodiment 47: A PPARγ agonist (e.g. TZD) for use in the treatment of steroid-dependent nephrotic syndrome (SDNS) in a subject (e.g. pediatric subject) in need thereof.

Embodiment 48: A PPARγ agonist (e.g. TZD) for use in the treatment of frequent relapsing nephrotic syndrome (FRNS) in a subject (e.g. pediatric subject) in need thereof.

Embodiment 49: A PPARγ agonist (e.g. TZD) for use according to any one of Embodiments 46-48, wherein the subject (e.g. pediatric subject) has focal segmental glomerulosclerosis.

Embodiment 50: A PPARγ agonist (e.g. TZD) for use according to any one of Embodiments 46-48, wherein the subject (e.g. pediatric subject) has collapsing focal segmental glomerulosclerosis.

Embodiment 51: A PPARγ agonist (e.g. TZD) for use according to any one of Embodiments 46-48, wherein the subject (e.g. pediatric subject) has minimal change nephrotic syndrome.

Embodiment 52: A PPARγ agonist (e.g. TZD) for use according to any one of Embodiments 46-51, wherein the subject (e.g. pediatric subject) is also being treated with one or more immunosuppressive medications.

Embodiment 53: A PPARγ agonist (e.g. TZD) for use according to Embodiment 52, wherein an immunosuppressive medication is a glucocorticoid, such as prednisone, prednisolone, methylprednisolone and dexamethasone.

Embodiment 54: A PPARγ agonist (e.g. TZD) for use according to Embodiment 52 or 53, wherein the one or more immunosuppressive medications is/are administered at a reduced dose compared to standard therapy.

Embodiment 55: A PPARγ agonist (e.g. TZD) for use according to any one of Embodiments 52-54, wherein administering the PPARγ agonist (e.g. TZD) produces a immunosuppressive medication sparing effect.

Embodiment 56: A PPARγ agonist (e.g. TZD) for use according to any one of Embodiments 46-55, wherein the PPARγ agonist (e.g. TZD) is selected from the group consisting of pioglitazone, rosiglitazone, lobeglitazone, ciglitazone, darglitazone, englitazone, netoglitazone, rivoglitazone, troglitazone, and balaglitazone.

Embodiment 57: A PPARγ agonist (e.g. TZD) for use according to any one of Embodiments 46-55, wherein the PPARγ agonist (e.g. TZD) is pioglitazone.

Embodiment 58: A PPARγ agonist (e.g. TZD) for use according to Embodiment 57, wherein pioglitazone is administered orally to the subject (e.g. pediatric subject) at a dose of about 15-45 mg QD (i.e., per day).

Embodiment 59: A composition for use in the treatment of nephrotic syndrome (NS) in a subject (e.g. pediatric subject) in need thereof comprising a PPARγ agonist (e.g. TZD), optionally together with one or more pharmaceutically acceptable carriers and/or excipients.

Embodiment 60: A composition for use in the treatment of steroid-dependent nephrotic syndrome (SDNS) in a subject (e.g. pediatric subject) in need thereof comprising a PPARγ agonist (e.g. TZD), optionally together with one or more pharmaceutically acceptable carriers and/or excipients.

Embodiment 61: A composition for use in the treatment of frequent relapsing nephrotic syndrome (FRNS) in a subject (e.g. pediatric subject) in need thereof comprising a PPARγ agonist (e.g. TZD), optionally together with one or more pharmaceutically acceptable carriers and/or excipients.

Embodiment 62: A composition for use according to any one of Embodiments 59-61, wherein the subject (e.g. pediatric subject) has focal segmental glomerulosclerosis.

Embodiment 63: A composition for use according to any one of Embodiments 59-61, wherein the subject (e.g. pediatric subject) has collapsing focal segmental glomerulosclerosis.

Embodiment 64: A composition for use according to any one of Embodiments 59-61, wherein the subject (e.g. pediatric subject) has minimal change nephrotic syndrome.

Embodiment 65: A composition for use according to any one of Embodiments 55-64, wherein the subject (e.g. pediatric subject) is also being treated with one or more immunosuppressive medications.

Embodiment 66: A composition for use according to Embodiment 65, wherein an immunosuppressive medication is a glucocorticoid, such as prednisone, prednisolone, methylprednisolone and dexamethasone.

Embodiment 67: A composition for use according to Embodiment 65 or 66, wherein the one or more immunosuppressive medications is/are administered at a reduced dose compared to standard therapy.

Embodiment 68: A composition for use according to any one of Embodiments 65-67, wherein administering the PPARγ agonist (e.g. TZD) produces a immunosuppressive medication sparing effect.

Embodiment 69: A composition for use according to any one of Embodiments 59-68, wherein the TZD is selected from the group consisting of pioglitazone, rosiglitazone, lobeglitazone, ciglitazone, darglitazone, englitazone, netoglitazone, rivoglitazone, troglitazone, and balaglitazone.

Embodiment 70: A composition for use according to any one of Embodiments 59-68, wherein the TZD is pioglitazone.

Embodiment 71: A composition for use according to Embodiment 70, wherein pioglitazone is administered orally to the subject (e.g. pediatric subject) at a dose of about 15-45 mg QD (i.e., per day).

### Administration and Formulation

The pharmaceutical compositions of the present invention comprise a thiazolidinedione (e.g., pioglitazone) and in some embodiments a glucocorticoid as active ingredients, or pharmaceutically acceptable salts thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the present compounds can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The present compounds may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethypiperideine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

The data obtained from the cell culture assays and animal studies can be used in formulating a range of dosage for use in humans. The dosage may vary depending upon the dosage form employed and the route of administration. A dose may be formulated in animal models to achieve a circulating plasma concentration range that includes the IC₅₀ (i.e., the concentration of the test compound which achieves a half-maximal inhibition of symptoms). Such information can be used to more accurately determine useful doses in humans. Levels in plasma may be measured, for example, by high performance liquid chromatography.

As defined herein, a therapeutically effective amount of the thiazolidinedione or glucocorticoid (i.e., an effective dosage) ranges from 0.001 to 30 mg/kg body weight, preferably 0.01 to 25 mg/kg body weight, more preferably 0.1 to 20 mg/kg body weight, and even more preferably 1 to 10 mg/kg, 2 to 9 mg/kg, 3 to 8 mg/kg, 4 to 7 mg/kg, or 5 to 6 mg/kg body weight. The active compounds can be administered one time per week for between 1 to 10 weeks, preferably between 2 to 8 weeks, more preferably between 3 to 7 weeks, and even more preferably for 4, 5, or 6 weeks. The skilled artisan will appreciate that certain factors may influence the dosage and timing required to effectively treat a mammal including, but not limited to, the severity of the disease or disorder, previous treatments, the general health and/or age of the mammal, and other diseases present. Moreover, treatment of a mammal with a therapeutically effective amount of thiazolidinedione (e.g., pioglitazone) and a glucocorticoid can include a single treatment or, preferably, can include a series of treatments.

The present invention is illustrated by the following example. It is to be understood that the particular examples, materials, amounts, and procedures are to be interpreted broadly in accordance with the scope and spirit of the invention as set forth herein.

### Example

In the following clinical studies, the efficacy and safety of adding pioglitazone to reduce proteinuria in children with NS was examined.

### Patient Inclusion

All children with NS identified worldwide to have been treated with pioglitazone were included, with no patients excluded. Each patient had pioglitazone added to their other NS medications at the discretion of their treating pediatric nephrologist. All patient records were reviewed in a de-identified manner, with prior ethics committee approval at each institution. Data were analyzed based on defined time periods before and after the addition of pioglitazone. Patients were defined as having responded to pioglitazone (i.e., Responders) if their urine protein to creatinine ratio (UPCR, mg/mg) decreased from the period 0-1 month pre-pioglitazone to the period 0-1 month post-pioglitazone. Glomerular filtration rate estimation (eGFR) in patients <18 years was calculated using the revised Schwartz equation. In patients >18 years, eGFR was calculated as the mean of the revised Schwartz and CKD-EPI creatinine equations.

### Determination of Overall Patient Immunosuppression

For each patient, a Total Immunosuppression Score (TIS) (see Table 3) was calculated to quantitate overall immunosuppression. For each class of immunosuppressive drug administered during a given time period, a score of 0-4 was assigned based on the percentage of a full dose for each patient's body size (0=0%; 1=<25%; 2=≥25-<50%; 3=≥50-<75%: 4=≥75%).

### Data Analysis

Quantitative data, such as patient age, duration of NS, pioglitazone dosage, and outcomes of UPCR, TIS, and serum albumin, were summarized as means (standard errors of mean), while categorical data such as renal survival status were summarized as frequency (percent). Due to a small sample size, Responders and Non-responders were compared using Wilcoxon Rank Sum tests on quantitative variables and Fisher's exact tests on categorical variables. For both Responders and Non-responders, pre-pioglitazone to post-pioglitazone initiation changes in UPCR, TIS, and serum albumin levels were analyzed using Wilcoxon Signed Rank tests. Numbers of hospitalizations pre-pioglitazone and post-pioglitazone initiation were analyzed and compared using Poisson distributions. Statistical significance was assessed at both the 0.05 and 0.10 levels. All analyses were conducted using SPSS software ver. 26 (IBM, Armonk, NY, USA).

### Results

### Clinical Outcomes

Patient characteristics, including age at NS onset, age at pioglitazone treatment, gender, ethnicity, and clinical and histologic disease characteristics, are shown in Table 1. On initial biopsy 4 patients had MCNS, 2 had FSGS, and 2 had cFSGS. Two MCNS patients progressed to FSGS on second biopsies. Genetic testing was performed in Patient 1, with no pathogenic genetic variants identified. The pre-pioglitazone steroid response patterns included 3 patients with SDNS, 3 with SRNS, and 2 in whom PIO was started simultaneously with steroids.

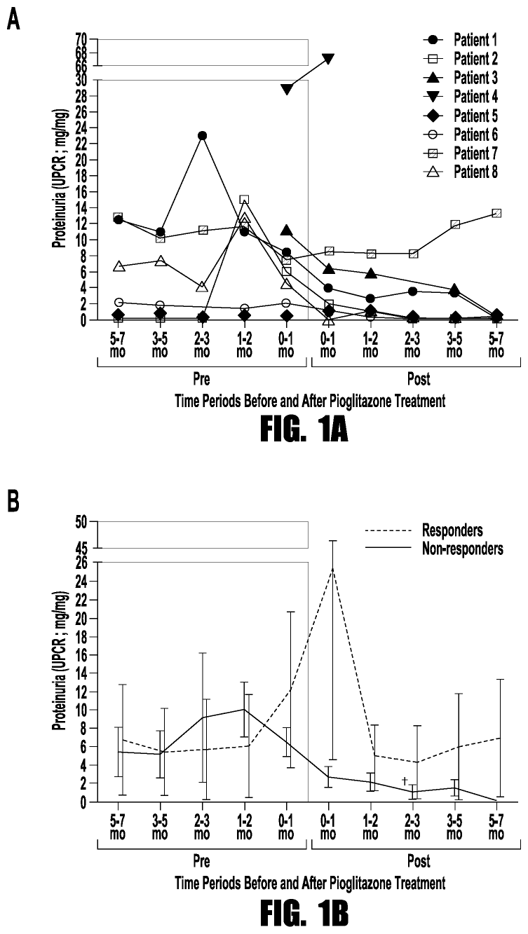

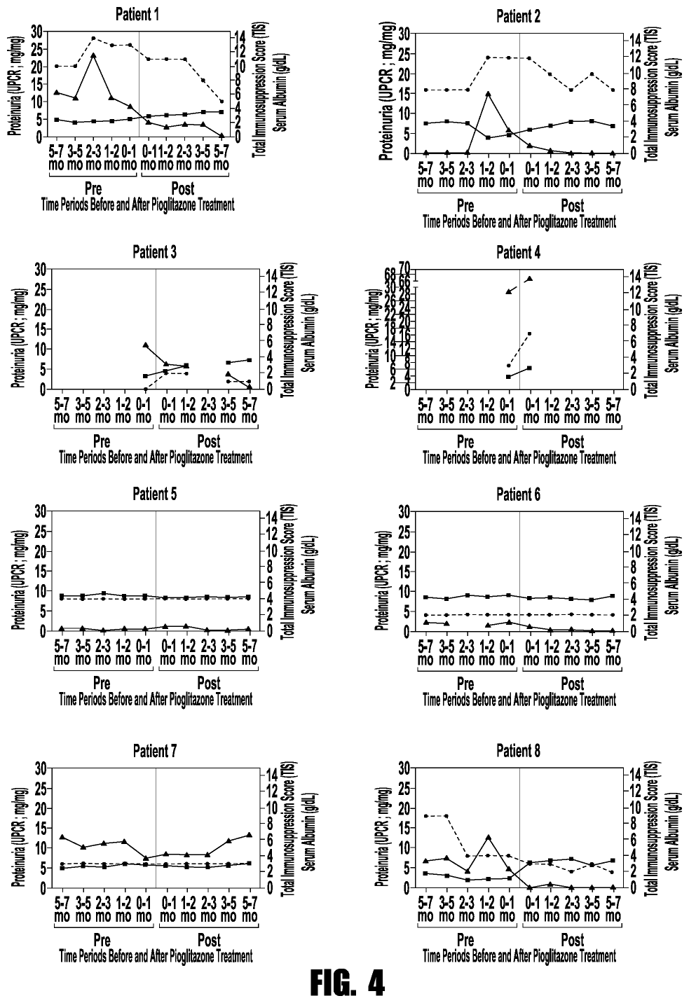

Clinical data, including proteinuria, serum albumin, and TIS for all 8 patients from 5-7 months pre-pioglitazone until 5-7 months post-pioglitazone introduction are shown in FIG. 4. Five of 8 pioglitazone-treated patients (63%) had prompt and significant reductions in proteinuria (Responders; Cases 1, 2, 3, 6, and 8), while 3 patients had worsened proteinuria after pioglitazone initiation (Non-responders; Patients 4, 5, and 7)—see FIG. 1. The UPCR in Responders decreased from a mean of 6.5 mg/mg (range 2.2-11.2) to 2.7 mg/mg (range 0.1-6.4) within the first month of pioglitazone treatment (62% mean reduction; range 43-98%; P=0.04). By 5-7 months post-pioglitazone the UPCR had dropped even further to a mean of 0.2 mg/mg (97% mean reduction; range 0.06-0.5; P=0.04)—see Table 2. Of the 5 Responders, 2 had MCNS, 1 had MCNS progressing to FSGS, 1 had FSGS, and 1 had cFSGS (Table 1).

Among pioglitazone Non-responders, Patient 4's UPCR increased from 28.8 to 67.0 mg/mg (FIGS. 1A and 4), although the post-pioglitazone urine was obtained soon after an IV albumin infusion, and he progressed rapidly to end stage kidney disease (ESKD) 6.5 weeks after presentation and 3.5 weeks after biopsy and PIO initiation. The other Non-responders (Patients 5 and 7) had 24% and 77% increases in proteinuria, respectively, at 5-7 months post-pioglitazone (FIGS. 1A and 4).

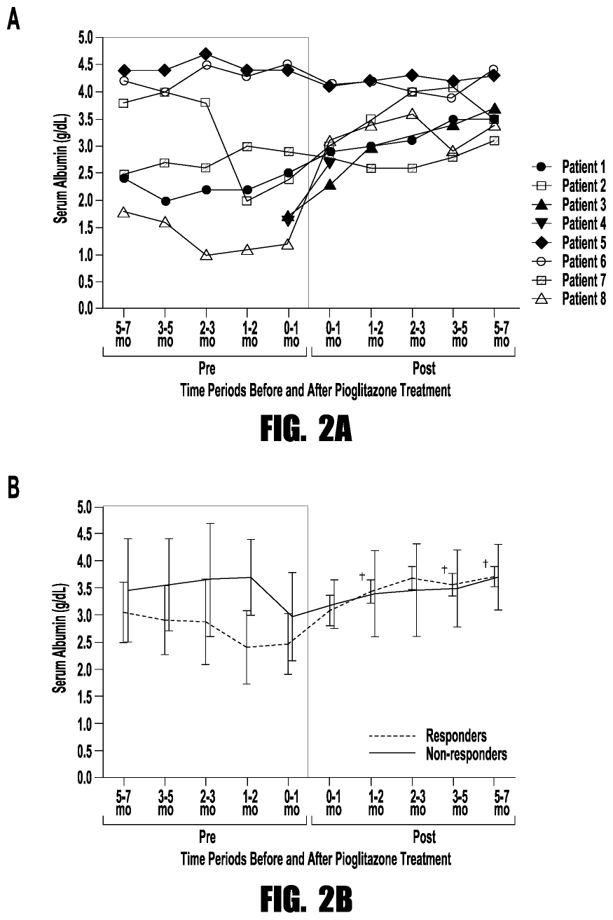

Among Responders, the mean serum albumin 0-1-month pre-pioglitazone was 2.5 g/dL (range 1.2-4.5 g/dL), increased to 3.1 g/dL 0-1 month post-pioglitazone (45% mean increase; range −9-158%; P>0.10), and further increased to 3.7 g/dL 5-7 months post-pioglitazone (77% mean increase; range −2-183%; P=0.08)—see FIG. 2. Among Non-responders, the increased serum albumin in Patient 4 was thought to be attributable not to reduced proteinuria but to a progressive decline in kidney function.

The Total Immunosuppression Score (TIS) (Table 3) has been previously used to normalize overall immunosuppressive medication exposure (and thus risk for drug-induced toxicity) in patients treated with a variety of immunosuppressive medications at varying doses over time. Among Responders, the dramatic reductions in proteinuria post-pioglitazone enabled commensurate decreases in TISs during pioglitazone treatment (FIG. 3), which decreased from a mean of 6.2 at 0-1-month pre-pioglitazone to 3.6 at 5-7 months post-pioglitazone, representing a 42% (P >0.10) decrease in overall exposure to immunosuppressive medications. In contrast, among Non-responders the mean TIS remained generally stable at approximately 3.5 before and after pioglitazone treatment (FIG. 3).

All 3 Non-responders (100%) had some degree of renal dysfunction prior to pioglitazone initiation. Patient 4 had cFSGS and AKI at presentation (eGFR 51 ml/min/1.73 m²), which progressed to ESKD within 3.5 weeks. Patient 5, with NS for 13 years, had CKD Stage 2 at 0-1-month pre-PIO (eGFR 86 ml/min/1.73 m²). Patient 7, with NS for 10 years, had CKD Stage 3 at 0-1-month pre-PIO (eGFR 44 ml/min/1.72 m²). In contrast, only 1 of 5 Responders (20%; Patient 6) had renal dysfunction at the time of PIO initiation (eGFR 81 ml/min/1.73 m²).

Safety concerns have previously been raised about the potential for TZDs to worsen edema through their renal tubular action, especially among patients with concurrent heart disease. However, the 8 patients here required 13 hospitalizations for IV albumin infusions to manage NS-related edema in the 5-7 months pre-pioglitazone treatment, but only 1 hospitalization in the 5-7 months post-pioglitazone treatment. In addition, among the 5 Responders the required hospitalizations dropped from 11 pre-pioglitazone treatment and none post-pioglitazone treatment (P<0.01). Thus, despite concerns about worsening edema among adults treated with TZDs, no children with NS (0%) had worsening edema after pioglitazone introduction. Instead, the dramatic improvements in proteinuria and edema in most patients led to a marked decrease in required hospitalizations. In addition, no children with NS were noted to have pioglitazone-related hypoglycemia during the observation period.

TABLE 1
Patient Characteristics

| | Patient Number | | | | | | | |
|---|---|---|---|---|---|---|---|---|
| | 1 | 2 | 3 | 4 | 5 | 6 | 7 | 8 |
| Age at NS | 4 | 5 | 13 | 6 | 8 | 7 | 4 | 4 |
| Presentation | | | | | | | | |
| (years) | | | | | | | | |
| Age at PIO | 4 | 9 | 13 | 6 | 20 | 27 | 14 | 11 |
| Initiation | | | | | | | | |
| (years) | | | | | | | | |
| Gender | Male | Male | Female | Male | Male | Female | Female | Male |
| Ethnicity | AA/NA | AA | Caucasian | Caucasian | Chinese | Malay | Chinese | Japanese |
| 1^{st} Renal | MCNS | MCNS | cFSGS | cFSGS | MCNS | FSGS | FSGS | MCNS |
| Biopsy | | | | | | | | |
| 2^{nd} Renal | FSGS | — | — | — | FSGS | — | — | MCNS |
| Biopsy | | | | | | | | |
| Steroid | SRNS | SDNS | * | * | SDNS | SRNS | SRNS | SDNS |
| Response | | | | | | | | |
| Pattern | | | | | | | | |

PIO = pioglitazone; AA = African American; NA = Native American; MCNS = Minimal change nephrotic syndrome; FSGS = focal segmental glomerulosclerosis; cFSGS = collapsing FSGS; SRNS = steroid resistant nephrotic syndrome; SDNS = steroid dependent nephrotic syndrome.
(* = Steroid response pattern could not be ascertained as PIO was started nearly simultaneously with prednisone; — = no second biopsy performed)

TABLE 2
Pioglitazone Treatment and Outcomes

| | Responders | | | | | | Non-responders | | | | |
|---|---|---|---|---|---|---|---|---|---|---|---|
| | Patient Number | | | | | | | | | | |
| | | | | | | Mean | | | | Mean | P- |
| | 1 | 2 | 3 | 6 | 8 | (SEM) | 4 | 5 | 7 | (SEM) | Value |
| Age at | 4 | 9 | 13 | 27 | 11 | 12.8 | 6 | 20 | 14 | 13.3 | NS |
| PIO Start | | | | | | (3.85) | | | | (4.06) | |
| (years) | | | | | | | | | | | |
| Duration | 1.1 | 1 | 0.25 | 18 | 7.8 | 5.63 | 0.17 | 13 | 10 | 7.72 | NS |
| of NS | | | | | | (3.38) | | | | (3.87) | |
| before Pio | | | | | | | | | | | |
| (years) | | | | | | | | | | | |
| Starting | 15 | 15 | 15 | 30 | 30 | 21.0 | 7.5 | 45 | 15 | 22.5 | NS |
| PIO Dose | | | | | | (3.67) | | | | (11.46) | |
| (mg/d) | | | | | | | | | | | |
| Peak PIO | 30 | 15 | 30 | 30 | 30 | 27.0 | 7.5 | 45 | 30 | 27.5 | NS |
| Dose | | | | | | (3.00) | | | | (10.9) | |
| (mg/d) | | | | | | | | | | | |
| Pre-PIO | 8.5 | 6 | 11.2 | 2.16 | 4.66 | 6.50 | 28.8 | 0.46 | 7.5 | 12.3 | NS |
| UPCR (0-1 | | | | | | (1.56) | | | | (8.52) | |
| mo; mg/mg) | | | | | | | | | | | |
| Post-PIO | 4 | 2 | 6.4 | 1.09 | 0.08 | 2.71 | 67 | 1.1 | 8.5 | 25.5 | NS |
| UPCR (0-1 | | | | | | (1.13) * | | | | (20.8) | |
| mo; mg/mg) | | | | | | | | | | | |
| % UPCR | 52.9 | 66.7 | 42.9 | 49.5 | 98.3 | 62.1 | −133 | −139 | −13.3 | −95.0 | 0.036 |
| Reduction | | | | | | (9.85) | | | | (40.9) | |
| (0-1 mo) | | | | | | | | | | | |
| Post-PIO | 0.2 | 0.12 | 0.5 | 0.11 | 0.06 | 0.20 | ESRD | 0.57 | 13.3 | 6.94 | 0.095 |
| UPCR (5-7 | | | | | | (0.08) * | | | | (5.20) | |
| mo; mg/mg) | | | | | | | | | | | |
| % UPCR | 97.6 | 98.0 | 95.5 | 94.9 | 98.7 | 97.0 | NA | −23.9 | 77.3 | −50.6 | 0.095 |
| Reduction | | | | | | (0.74) | | | | (21.8) | |
| (5-7 mo) | | | | | | | | | | | |
| Pre-PIO | 13 | 12 | 0 | 2 | 4 | 6.20 | 3 | 4 | 3 | 3.33 | NS |
| TIS (0-1 mo) | | | | | | (2.65) | | | | (0.33) | |
| Post-PIO | 11 | 12 | 2 | 2 | 3 | 6.00 | 7 | 4 | 3 | 4.67 | NS |
| TIS (0-1 mo) | | | | | | (2.26) | | | | (1.20) | |
| % TIS | 15.4 | 0.00 | −∞ | 0.00 | 25.0 | −∞ | −133 | 0.00 | 0.00 | −44.4 | NS |
| reduction | | | | | | | | | | (44.4) | |
| (0-1 mo) | | | | | | | | | | | |
| Post-PIO | 5 | 8 | 1 | 2 | 2 | 3.60 | ESRD | 4 | 3 | 3.50 | NS |
| TIS (5-7 mo) | | | | | | (1.29) | | | | (0.41) | |
| % TIS | 61.5 | 33.3 | −∞ | 0.00 | 50.0 | −∞ | NA | 0.00 | 0.00 | 0.00 | NS |
| reduction | | | | | | | | | | (0.00) | |
| (5-7 mo) | | | | | | | | | | | |
| Pre-PIO S | 2.5 | 2.4 | 1.7 | 4.5 | 1.2 | 2.46 | 1.6 | 4.4 | 2.9 | 2.97 | NS |
| Alb (0-1 | | | | | | (0.56) | | | | (0.81) | |
| mo; mg/dL) | | | | | | | | | | | |
| Post-PIO S | 2.9 | 3 | 2.3 | 4.1 | 3.1 | 3.08 | 2.7 | 4.1 | 2.8 | 3.20 | NS |
| Alb (0-1 | | | | | | (0.29) | | | | (0.45) | |
| mo; mg/dL) | | | | | | | | | | | |
| % S Alb | 16.0 | 25.0 | 35.3 | −8.89 | 158 | 45.2 | 68.8 | −6.82 | −3.45 | 19.5 | NS |
| increase | | | | | | (29.2) | | | | (24.7) | |
| (0-1 mo) | | | | | | | | | | | |
| Post-PIO S | 3.5 | 3.5 | 3.7 | 4.4 | 3.4 | 3.70 | ESRD | 4.3 | 3.1 | 3.70 | NS |
| Alb (5-7 | | | | | | (0.18) † | | | | (0.49) | |
| mo; mg/dL) | | | | | | | | | | | |
| % S Alb | 40.0 | 45.8 | 118 | −2.22 | 183 | 76.9 | — | −2.27 | 6.90 | 2.31 | NS |
| increase | | | | | | (32.8) | | | | (3.74) | |
| (5-7 mo) | | | | | | | | | | | |
| Renal | yes | yes | yes | yes | yes | 100% | no | yes | no | 33.3% | NS |
| Survival | | | | | | survival | | | | survival | |

PIO = pioglitazone;
UPCR = urine protein to creatinine ratio;
TIS = total immunosuppression score;
S Alb = serum albumin
(* = P < 0.05 vs. 0-1 mo pre-PIO;
† = P < 0.10 vs. 0-1 mo pre-PIO;
∞ = infinitely large;
NA = numeric value not available;
NS = P ≥ 0.10 Responders vs. Non-responders

TABLE 3
Total Immunosuppression Score (TIS) Determination

| Immunosuppression | | | | | | | | |
|---|---|---|---|---|---|---|---|---|
| Score | Immunosuppressive Drugs | | | | | | | |
| (% of full dose) | PRED | DEX | TAC | CSA | Acthar ® | MMF | MA | RTX |
| 4 | ≥46 | ≥6.76 | ≥0.151 | >10 | ≥31 | ≥901 | ≥650 | <6 months |
| (≥75%) | mg/m2/d | mg/m2/d | mg/kg/d | mg/kg/d | units | mg/m2/d | mg/m2/d | after first |
| | | | | | BIW | | | dose (375 |
| | | | | | | | | mg/m2/dos |
| | | | | | | | | ex 1-4 doses) |
| 3 | 31-45 | 4.51-6.75 | 0.11-0.15 | 6.1-9.9 | 21-30 | 601-900 | 431-650 | n/a |
| (≥50-<75%) | mg/m2/d | mg/m2/d | mg/kg/d | mg/kg/d | units | mg/m2/d | mg/m2/d | |
| | | | | | BIW | | | |
| 2 | 16-30 | 2.26-4.5 | 0.051-0.1 | 4.1-6.0 | 11-20 | 301-600 | 221-430 | 6-9 months |
| (≥25-<50%) | mg/m2/d | mg/m2/d | mg/kg/d | mg/kg/d | units | mg/m2/d | mg/m2/d | after first |
| | | | | | BIW | | | dose (375 |
| | | | | | | | | mg/m2/dos |
| | | | | | | | | ex 1-4 doses) |
| 1 | ≤15 | ≤2.25 | ≤0.05 | <4 | ≤10 | ≤300 | ≤220 | n/a |
| (<25%) | mg/m2/d | mg/m2/d | mg/kg/d | mg/kg/d | units | mg/m2/d | mg/m2/d | |
| | | | | | BIW | | | |
| 0 | 0 | 0 | 0 | 0 | 0 units | 0 | 0 | >9 months |
| (0%) | mg/m²/d | mg/m²/d | mg/kg/d | mg/kg/d | BIW | mg/m²/d | mg/m²/d | after first |
| | | | | | | | | dose (375 |
| | | | | | | | | mg/m²/dos |
| | | | | | | | | ex 1-4 doses) |
| TOTAL IMMUNO- | A | B | C | D | E | F | G | H |
| SUPPRESSION | | | | | | | | |
| SCORE (TIS) = | | | | | | | | |
| (Sum of A-H) | | | | | | | | |

TIS = Total Immunosuppression Score; PRED = Prednisone; DEX = Dexamethasone; TAC = Tacrolimus; CSA = Cyclosporine A; MMF = Mycophenolate mofetil; MA = mycophenolic acid; RTX = Rituximab.
Potential TIS Scores: Maximal score = 4 points per immunosuppressive drug.

To summarize the results, following pioglitazone introduction the majority of patients (63%, Responders) experienced significant decreases in proteinuria (97% after 6 months), increases in serum albumin (from 2.5 to 3.7), reductions in hospitalizations for IV albumin infusions and diuresis (100%), and decreases in total immunosuppression (42%). Importantly, no patients experienced any adverse events attributable to pioglitazone during a collective 136 months of pioglitazone drug exposure.

The following are the results obtained for each of the eight subjects discussed hereinabove:

### Subject 1

A 4-Year-Old Male was Diagnosed with SRNS and Renal Biopsy Showed Minimal Change NS (MCNS). Despite ongoing treatment with prednisone (PRED), lisinopril, tacrolimus (TAC), mycophenolate (MMF), losartan, rituximab (RTX), and repository corticotropin injection (Acthar®) he still required twice-weekly hospital admissions for diuresis of anasarca. To try to reduce hospitalizations and proteinuria, pioglitazone (15 mg orally QD) was added and increased after 4 weeks to 30 mg orally QD. His UPCR decreased dramatically from 8.5 to 4 (53% reduction) over the next 1 month and to 0.2 (98% reduction) over the next 6 months, which enabled his cumulative immunosuppression (measured as TIS) to be decreased by 15% over the next 1 month and by 62% over the next 6 months. He maintained normal renal function. No side effects attributable to pioglitazone were noted throughout his 7-month PIO treatment.

### Subject 2

A 5-year-old male was diagnosed with SDNS and renal biopsy showed MCNS. Both MMF and PRED (30 mg/d) were required to maintain remission. After starting on pioglitazone (15 mg orally QD) for 16 weeks he achieved full remission and PRED was tapered to 5 mg orally QD. His UPCR decreased dramatically from 6 to 2 (67% reduction) over the next 1 month and to 0.12 (98% reduction) over the next 6 months, which enabled his cumulative immunosuppression (measured as TIS) to be decreased by 0% over the next 1 month and by 33% over the next 6 months. He maintained normal renal function with no side effects attributable to pioglitazone noted throughout his 4-month pioglitazone treatment.

### Subject 3

A 13-year-old obese female (BMI 33.6) was diagnosed with NS and renal biopsy showed collapsing focal segmental glomerulosclerosis (cFSGS). She was treated with PRED (40 mg/d), valsartan (80 mg/d), simvastatin (20 mg/d), and pioglitazone (15 mg orally QD). Her UPCR decreased dramatically from 11.2 to 6.4 (43% reduction) over the next 1 month and to 0.5 (96% reduction) over the next 6 months. Her cumulative immunosuppression (measured as TIS) increased from 0 to 2 over the next 1 month and to 1 over the next 6 months. Prednisone was then decreased to 20 mg/d and pioglitazone increased to 30 mg orally QD. After 6 months of pioglitazone treatment the UPCR declined further to 0.5 (95% reduction). No side effects attributable to pioglitazone were noted throughout her 19-month course of pioglitazone treatment.

### Subject 4

A 6-year-old male was diagnosed with NS and renal biopsy showed cFSGS. Despite daily PRED, IV albumin infusions and furosemide, his renal function worsened to eGFR 23 ml/min/1.73 m². A renal ultrasound showed a solitary right kidney with increased echogenicity. His PRED was continued at 2 mg/kg/d, and pioglitazone (7.5 mg orally QD), pravastatin (20 mg orally QD), and 2 doses of RTX (375 mg/m²/dose 8 days apart) were added to his regimen. His UPCR increased from 28.8 to 67 (133% increase) over the next 1 month. His TIS increased from 3 to 7 (133% increase) over the next 1 month. Despite this his renal function continued to worsen, and he required hemodialysis three weeks later (6 weeks after initial presentation). During the 3 weeks of treatment with pioglitazone, no side effects attributable to pioglitazone were noted.

### Subject 5

An 8-year-old male was diagnosed with SDNS and renal biopsy showed MCNS. He responded to cyclophosphamide (CTX) but relapsed soon afterward. Subsequently, he responded to Cyclosporine A (CSA). Continued relapses (confounded by poor adherence) responded to increases in PRED and CSA doses. At 15 years old, a repeat biopsy showed FSGS, at which time CSA was changed to TAC. After drug-induced diabetes mellitus was diagnosed at age 16, his TAC was changed back to CSA, and he subsequently received 8 doses of RTX over the next 7 years, with only transient remissions after RTX infusions. At age 21, he was started pioglitazone 45 mg orally QD. His UPCR increased from 0.46 to 1.1 (139% increase) at 1-month post-PIO and remained at 0.57 (24% increase) at 6 months post-PIO. He remained on pioglitazone for 2.5 years but continued to have NS relapses. No side effects attributable to pioglitazone were noted throughout his pioglitazone treatment.

### Subject 6

A 7-year-old female was diagnosed SRNS and renal biopsy showed MCNS. She failed CTX induction but entered remission with CSA. She became PRED- and CSA-dependent and developed SDNS. At age 17, MMF was added to CSA, allowing PRED discontinuation. At age 19, a repeat biopsy showed FSGS with no CSA toxicity. CSA was switched to TAC, but she developed TAC toxicity requiring discontinuation. From ages 19-22, she received 6 doses of RTX, which led to partial remission. At age 25, she was diagnosed with diabetes mellitus and started on metformin. and subsequently glipizide and then insulin. At age 27, due to persistent proteinuria pioglitazone 30 mg orally QD was added to her other anti-diabetic medications. Her UPCR declined from 2.16 pre-pioglitazone to 1.09 (50% reduction) within 1 month post-pioglitazone, despite no change in her overall immunosuppression. Her UPCR decreased further to 0.11 (95% reduction) at 6 months post-pioglitazone and remained normal on MMF and pioglitazone, with a stable serum creatinine of 0.90 mg/dL. No side effects attributable to pioglitazone were noted throughout her 5-year course of pioglitazone treatment.

### Subject 7

A 4-year-old female presented with SRNS and renal biopsy showed FSGS. She was treated with PRED and CSA, inducing only partial remission, so MMF was added. Two years later. CSA was switched to TAC. At age 9, she received 4 doses of RTX and achieved complete remission. Her MMF was stopped and TAC continued. She then had a relapse which persisted despite restarting RTX and MMF. At age 14, pioglitazone was started at 15 mg PO pioglitazone and increased to 30 mg QD one month later. Her UPCR increased from 7.5 to 8.5 (13% increase) over the next 1 month and to 13.3 (77% increase) over the next 6 months, and pioglitazone was then stopped due to non-efficacy and concerns of possible association with new-onset bruising, which resolved. Her TIS remained stable at 3 over the next 6 months. Although her pre-pioglitazone renal function was impaired (eGFR 44 ml/min/1.73 m²), it remained stable during the 6 months of pioglitazone treatment, but progressed over the next 2.5 years to ESRD.

### Subject 8

A 4-year-old male presented with SDNS. His treatment included CSA, CTX, mizoribine, and MMF, which put him into remission at each relapse. Renal biopsies were performed at the ages of 6 years and 9 years, both of which showed MCNS. At age 10, two RTX infusions over eight months did not resolve his SDNS. LDL apheresis (12 treatments over 6 weeks) did not improve relapse frequency (7 per year), or to permit PRED taper. Pioglitazone was then started at 30 mg orally QD for 30 weeks. His UPCR decreased from 4.66 to 0.08 (98% reduction) within 1 month of pioglitazone initiation and to 0.06 (99% reduction) within 6 months. His TIS decreased from 4 to 3 (25% reduction) within 1 month and to 2 (50% reduction) within 6 months. He remained in complete remission for the following 5-7 months which enabled his PRED to be tapered from 25 to 10 mg/d. His renal function remained normal throughout this period. No side effects attributable to pioglitazone were noted throughout his 30-week course of pioglitazone treatment.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating nephrotic syndrome (NS) in a subject, comprising administering to said subject a therapeutically effective amount of a PPARγ agonist.

2. The method according to claim 1, wherein the nephrotic syndrome is steroid-dependent nephrotic syndrome (SDNS).

3. The method according to claim 1, wherein the nephrotic syndrome is frequent relapsing nephrotic syndrome (FRNS).

4. The method according to claim 1, wherein the subject has focal segmental glomerulosclerosis.

5. The method according to claim 1, wherein the subject is a pediatric subject.

6. The method according to claim 5, wherein the pediatric subject has an age from 1-12 years.

7. The method of claim 1, wherein the subject is also treated with one or more immunosuppressive medications.

8. The method according to claim 7, wherein an immunosuppressive medication is a glucocorticoid.

9. The method according to claim 8, wherein the glucocorticoid is selected from prednisone, prednisolone, methylprednisolone and dexamethasone.

10. The method according to claim 9, wherein the glucocorticoid is prednisone.

11. The method according to claim 7, wherein the PPARγ agonist and the one or more immunosuppressive medications act synergistically to treat NS.

12. The method according to claim 11, wherein the one or more immunosuppressive medications is/are administered at a reduced dose compared to standard therapy.

13. The method according to claim 1, wherein the PPARγ agonist is a thiazolidinedione (TZD).

14. The method according to claim 13, wherein the TZD is selected from the group consisting of pioglitazone, rosiglitazone, lobeglitazone, ciglitazone, darglitazone, englitazone, netoglitazone, rivoglitazone, troglitazone, and balaglitazone.

15. The method according to claim 13, wherein the TZD is pioglitazone.

16. The method according to claim 15, wherein pioglitazone is administered orally to a pediatric subject at a dose of about 15-45 mg per day.

17. The method according to claim 1, wherein administering the PPARγ agonist produces a reduction in proteinuria in the subject.

18. The method according to claim 17, wherein protein levels have been normalized within 5-7 months following initiation of PPARγ agonist therapy.

19. The method according to claim 1, wherein administering the PPARγ agonist increases the level of serum albumin in the subject.

20. The method according to claim 7, wherein administering the PPARγ agonist produces an immunosuppressive medication sparing effect.

21. The method according to claim 8, wherein administering a PPARγ agonist produces a glucocorticoid sparing effect.

22. The method according to claim 1, wherein NS is treated without any consequential adverse effects.

23. The method according to claim 1, wherein administering the PPARγ agonist produces an improvement in Patient Reported Outcomes (PROs), including measures of physical health, mental health, social health, or global health, in the subject.

24. The method according to claim 1, wherein treatment of NS further comprises an improvement in edema.
